(19) United States
(12) Patent Application Publication
(10) Pub. No.: US 2016/0310578 A1
(43) Pub. Date: Oct. 27, 2016

(54) METHOD OF ACCELERATING OSTEOGENESIS

(71) Applicants: Fuji Nihon Seito Corporation, Tokyo (JP); Yutaka Research Institute, Tokyo-to (JP)

(72) Inventors: Toshihiko INAGE, Tokyo (JP); Tadashi WADA, Shizuoka City (JP)

(21) Appl. No.: 15/137,654

(22) Filed: Apr. 25, 2016

(30) Foreign Application Priority Data
Apr. 27, 2015 (JP) 2015-089948

(51) Int. Cl. A61K 38/39; A61K 9/00; A61K 9/20; A61K 31/7048

(52) U.S. Cl. CPC A61K 38/39; A61K 31/7048; A61K 9/0053; A61K 9/20; A23L 1/304; A23L 1/305; A23V 2002/00

(57) ABSTRACT

A method for promoting osteogenesis including administering a therapeutically effective amount of collagen and a calcium absorption promoter to a subject.

### TECHNICAL FIELD

The present invention relates to a method for promoting osteogenesis. The present invention particularly relates to a method for promoting osteogenesis that includes administering a therapeutically effective amount of collagen and a calcium absorption promoter to a subject.

### BACKGROUND ART

The number of osteoporosis patients is increasing as the population of the elderly increases. Osteoporosis is a disease in which bones become brittle due to reduced amount of bone. Bones are constantly being re-formed, and for a healthy individual, amount of bone decreased by bone resorption and amount of bone increased by bone formation are substantially equal, and the total amount of bone in a body is kept constant. For elderly people, amount of bone increased by bone formation becomes lower than amount of bone decreased by bone resorption, and thus they are highly likely to have osteoporosis.

Osteoporosis is closely related to hormone secretion, and often occurs after menopause due to decreasing female hormone, estrogen. According to the statistics on osteoporosis patients compiled by the Japanese Ministry of Health, Labour and Welfare in 2006, one in two women in their sixties and 7 in 10 women aged 70 and over suffered from osteoporosis. Also, the proportion of osteoporosis patients gradually increased among male population over 60, and 4 in 10 men aged 70 and over suffered from the disease. It is estimated that there are currently approximately 8 million women and approximately 2 million men, for a total of 10 million people, suffering from osteoporosis in Japan.

The health care cost of, for example, bone fracture in osteoporosis patients is increasing year after year, and is becoming not only a medical problem, but a societal problem as well. The health care cost required for treatment of osteoporosis in the U.S. has grown to 14 trillion yen.

Another problem in osteoporosis is “locomotive syndrome”, which is induced by bones losing density and weakening. From 2006 to 2010, the Japanese Ministry of Health, Labour and Welfare directed a domestic project to find a solution for locomotive syndrome, and although a great deal of research was conducted, no method for treatment has been developed as of yet.

Bone tissue is remodeled by constant repetition of osteogenesis by osteoblasts and bone resorption by osteoclasts. Normally, bone remodeling is regulated by coordination between osteoblasts and osteoclasts and by hormones and growth factors that regulate bone growth, so that amount of bone and the calcium contained in bone are held in homeostasis.

For this reason, calcium preparations and calcitonin, which is a parathyroid hormone that promotes osteogenesis, are widely used in the treatment of osteoporosis. However, a satisfactory effect has not been obtained so far.

Furthermore, bisphosphonate, which suppress the bone resorption action of osteoclasts, have been used recently as a clinical therapy for osteoporosis. Bone tissue is constantly being re-formed by repetition of resorption and formation, but bisphosphonate preparations suppress bone resorption by inhibiting bone resorption action. Therefore, since there is no resorption of old bone that should be actually be resorbed, there are cases in which sequestrum is formed. Additionally, in patients who are administered with bisphosphonate preparations, there is a tendency for bone regeneration to be prolonged because remodeling of bone tissue is reduced.

Bone tissue contains approximately 64% inorganic substance and the remainder is organic substance such as collagen and osteonectin. When bone tissue is formed, inorganic substance primarily containing calcium and magnesium deposits in the framework made from collagen and non-collagen proteins such as osteonectin and bone sialoprotein.

The primary component of the inorganic substance of bone is formed from fine crystals called hydroxyapatite (Ca₁₀(PO₄)₆OH₂), and calcium is an important component for the formation of bone crystals. The calcium is adsorbed from the upper intestinal tract, but conventional calcium preparations for treating and preventing osteoporosis, when taken orally, are hardly absorbed from the small intestine without action of absorption promoter and end up being excreted to outside the body.

For this reason, an absorption promoter is required in order to absorb calcium from the small intestine. Difructose anhydride III (DFA III) is known as a calcium absorption promoter (JP2006-241057A, JP2005-247758A).

### SUMMARY OF INVENTION

A first aspect of the present invention is a method for promoting osteogenesis including administering a therapeutically effective amount of collagen and a calcium absorption promoter to a subject.

In the first aspect, the calcium absorption promoter may be difructose anhydride III.

In the first aspect, the amount of difructose anhydride III may be from approximately 1 mg to approximately 50,000 mg, or preferably from approximately 100 mg to approximately 1,000 mg.

In the first aspect, the amount of the collagen may be from approximately 100 mg to approximately 80,000 mg, and preferably from approximately 1,000 mg to approximately 10,000 mg.

In the first aspect, the collagen and calcium absorption promoter may be administered to a subject simultaneously or separately.

In the first aspect, the subject is a subject having symptoms of osteoporosis or a subject who may develop symptoms of osteoporosis in the future.

A second aspect of the present invention is a method for promoting osteogenesis including administering a composition containing a therapeutically effective amount of collagen and a calcium absorption promoter as active ingredients to a subject.

The collagen and calcium absorption promoter may be administered to a subject simultaneously or separately.

The subject includes a subject having symptoms of osteoporosis or a subject who may develop symptoms of osteoporosis in the future.

In the second aspect, the calcium absorption promoter may be difructose anhydride III.

In the second aspect, a content of difructose anhydride III in the composition may be from approximately 0.1 to approximately 30% by weight, or from approximately 0.5 to approximately 20% by weight.

In the second aspect, a content of collagen in the composition may be from approximately 0.1 to approximately 30% by weight, or from approximately 0.5 to approximately 20% by weight.

In the second aspect, the administration may be oral administration.

In the second aspect, the composition may be a tablet.

In the second aspect, the composition may be a supplement.

In the second aspect, both the collagen and calcium absorption promoter may be contained in a single composition, e.g., in one tablet, or each of them may be contained in separate compositions, e.g., the collagen in one tablet and the calcium absorption promoter in another tablet. In the latter case, the separate compositions may be administered to a subject simultaneously or non-simultaneously.

In the second aspect, the subject includes a subject having symptoms of osteoporosis or a subject who may develop symptoms of osteoporosis in the future.

The present invention may provide a method for promoting osteogenesis, which promotes calcium absorption and effectively generates bones with the absorbed calcium. The method of the present invention enables prevention and treatment of osteoporosis.

The collagen used herein is not limited as long as it is a collagen peptide that can be ingested by humans. Examples of the collagen include pig collagen peptides, bovine collagen peptides, fish collagen peptides, sheep collagen peptides, and the like. Among these, fish collagen peptides are preferred. The amount used is, for example, from approximately 100 mg to approximately 80,000 mg, and preferably from approximately 1,000 mg to approximately 10,000 mg, as a daily dose.

Examples of the calcium absorption promoter include citrate-malate (CCM), casein phosphopeptide (CPP), lactose, fructooligosaccharide, DFA III, and the like. These calcium absorption promoters may be used each alone or in combinations of two or more types. The amount used is, for example, from approximately 1 mg to approximately 50,000 mg, and preferably from approximately 100 mg to approximately 1,000 mg, as a daily dose.

In the method of the present invention, vitamins, minerals, and the like may also be administered in addition to the collagen peptide and the calcium absorption promoter.

Examples of the vitamins include vitamin A, vitamin Bs, vitamin C, vitamin D, vitamin E, and vitamin Ks. The amount of vitamins added may be, for example, for vitamin A, from approximately 1 mg to approximately 10,000 mg, and preferably approximately 15 mg; for vitamin C, from approximately 1 mg to approximately 10,000 mg, and preferably 100 mg; for vitamin K₂ (0.2%), from approximately 1 mg to approximately 10,000 mg, and preferably approximately 25 mg; for vitamin B1 (thiamine nitrate), approximately 1 mg to approximately 10,000 mg, and preferably approximately 1 mg; for vitamin B6 (pyridoxine hydrochloride), from approximately 1 mg to approximately 10,000 mg, and preferably approximately 12 mg, as a daily dose.

Examples of the minerals include calcium citrate, calcium carbonate, calcium hydrogen phosphate, magnesium carbonate, monobasic calcium phosphate, dibasic calcium phosphate, tribasic calcium phosphate, calcium octaphosphate, amorphous calcium phosphate, hydroxyapatite, calcium hydroxide, and the like. The amount of minerals added may be, for example, for calcium citrate, from approximately 1 mg to approximately 10,000 mg, and preferably approximately 50 mg; for calcium carbonate, from approximately 1 mg to approximately 10,000 mg, and preferably 600 mg; for calcium hydrogen phosphate, from approximately 1 mg to approximately 10,000 mg, and preferably approximately 200 mg; and for magnesium carbonate, from approximately 1 mg to approximately 10,000 mg, and preferably approximately 300 mg, as a daily dose.

In addition to vitamins and minerals, malic acid, polyglutamic acid, glucosamine hydrochloride, chondroitin, β-cryptoxanthin, hyaluronic acid, and the like may be added. The amount added may be, for example, for malic acid, from approximately 1 mg to approximately 10,000 mg, and preferably approximately 30 mg; for polyglutamic acid, from approximately 1 mg to approximately 10,000 mg, and preferably 20 mg; for glucosamine hydrochloride, from approximately 1 mg to approximately 10,000 mg, and preferably approximately 800 mg; for chondroitin, from approximately 1 mg to approximately 10,000 mg, and preferably approximately 400 mg, as a daily dose.

The composition of the present invention may be orally administered preparation or a supplement, which can be orally administered. Examples of such a preparation include tablets such as orally disintegrating tablets, chewable tablets, and dispersible tablets, capsules, granules, powders, oral liquids, syrups, oral jellies, and the like.

When the composition of the present invention is prepared, pharmaceutically acceptable excipients, binders, lubricants, isotonizing agents, stabilizers, preservatives, flavorings, dissolution aids, emulsifiers, and the like may be further added.

The composition of the present invention may be produced by conventionally known methods. For example, for a tablet, it may be prepared by the following process. That is, a producing method which includes steps of weighting, primary mixing (homogeneously mixing of active ingredients, excipients, binders, disintegrating agents, and the like), granulating, sieving, secondary mixing (mixing of lubricants and the like), coating (as necessary), packaging, and the like.

The amount of collagen used in the method of the present invention is not particularly limited. For example, it may be from approximately 0.1 to approximately 30% by weight, preferably from approximately 0.5 to approximately 20% by weight, and more preferably from approximately 1 to approximately 10% by weight, of the total dose or the total amount of the composition.

The amount of calcium absorption promoter used in the method of the present invention is not particularly limited. For example, it may be from approximately 0.1 to approximately 30% by weight, preferably from approximately 0.5 to approximately 20% by weight, and more preferably from approximately 1 to approximately 10% by weight, of the total dose or the total amount of the composition.

### EXAMPLES

As a daily dose of the preparations used in the experiments, 20 tablets each containing the materials listed in Table 1 (called “tablet” hereinafter) were prepared.

TABLE 1

| | | |
|---|---|---|
| Collagen peptide | 5,000 | mg |
| CPP (casein phosphopeptide) | 130 | mg |
| Fructooligosaccharide | 450 | mg |
| Calcium citrate | 50 | mg |
| Malic acid | 30 | mg |
| Calcium carbonate | 600 | mg |
| Calcium hydrogen phosphate (anhydrous) | 200 | mg |
| Magnesium carbonate | 300 | mg |
| Polyglutamic acid (Meiji) | 20 | mg |
| Vitamin A | 15 | mg |
| Vitamin C | 100 | mg |
| Vitamin K2 (0.2%) | 25 | mg |
| Vitamin B1 (thiamine nitrate) | 1 | mg |
| Vitamin B6 (pyridoxine hydrochloride) | 12 | mg |
| Glucosamine hydrochloride (natural glucosamine) | 800 | mg |
| Chondroitin | 400 | mg |
| Fine silicon dioxide | 1,000 | mg |
| Crystalline cellulose | 660 | mg |
| Starch | 174 | mg |
| Sucrose fatty acid ester | 182 | mg |

Animal experiments were conducted using ovariectomized animals (OVX). The animals used were 48 female Wistar rats with body weight of approximately 350 g. They were used in the experiments after being preliminarily reared on standard feed (CE-2 manufactured by CLEA Japan, Inc.) for one week after undergoing ovariectomy. All experiments were approved by the Animal Experiment Ethics Committee of CLEA Japan, Inc. and were conducted according to the policies thereof. The standard feed CE-2 manufactured by CLEA Japan, Inc. contains 1.06 g of calcium per 100 g of the feed as calcium carbonate and calcium iodate (according to 2009 periodic analysis average values listed on the website of CLEA Japan, Inc.).

### Example 1

After preliminary feeding, six OVX rats were divided into two groups of three rats each. The experimental group was reared on CE-2 feed containing tablets in an amount equivalent to 3% collagen content, and the control group was reared on standard feed in which the collagen content was 3%. After being reared for one month, phenobarbital in a dose of 0.5 mg per 10 g of body weight was administered intraperitoneally to anesthetize the rats, and perfusion fixation was performed with 4% paraformaldehyde diluted with cacodylate buffer adjusted pH 7.4. After fixation, the articular head of femur was extracted, and X-ray images were taken using 3D CTR.

In the images taken by 3D CTR, the cortical bone was thicker and the mass and width of the cancellous bone were greater in the experimental group than in the control group. (FIG. 1)

Furthermore, bone area was measured using OsiriX imaging software (Table 2). The results demonstrated that the proportion of bone (bone mineral density) in the articular head was 11.4% greater in the experimental group than in the control group.

TABLE 2

| | Experimental group | Control group |
|---|---|---|
| Bone area (average) | 330.0 | 211.4 |
| μm² | | |
| Bone marrow | 245.9 | 249.5 |
| area (average) | | |
| μm² | | |
| Total area | 575.9 | 460.9 |
| μm² | | |
| Proportion of bone in | 57.3 | 45.9 |
| articular head (%) | | |

### Example 2

To confirm the effect in humans, five male or female volunteers aged 48 to 76 were administered 20 tablets per day for six months. The bone mineral density of the radial bone of the elbow was measured by an ultrasonic density measurement apparatus (CM-200 healthcare managing device) at the start of administration and after six months. The results are shown in Table 3 and FIG. 2.

TABLE 3

| | At start of | | |
|---|---|---|---|
| | administration | After 6 months | Rate of increase |
| | (g/cm²) | (g/cm²) | (%) |
| 48-year-old female | 33.5 | 40.2 | 12 |
| 61-year-old female | 33.1 | 36.4 | 10 |
| 69-year-old male | 30.2 | 35.3 | 17 |
| 75-year-old male | 27.2 | 32.4 | 19 |
| 76-year-old male | 26.3 | 31.3 | 19 |

From Table 3, bone mineral density increased by 10 to 19%. The average rate of increase was 15.4%.

### Example 3

The effect of DFA III on osteoblasts was examined using the osteoblastic cell line MC3T3-E1 isolated from human sarcoma.

First, 3×10⁵ cells of MC3T3-E1 were inoculated in 96 wells containing 90 μL of α-MEM medium containing penicillin (manufactured by Sigma) and streptomycin (manufactured by Sigma) (200 μg/mL). DFA III was added to the culture solution so as to result in concentrations of 10 mM, 50 mM, and 100 mM, and the cells were cultured at 37° C. at a CO₂ concentration of 5%. Culturing was performed for five wells each for each of the concentrations, and the number of the cells was counted on day 1, day 4, day 7, and day 14 after culturing.

When counting the number of the cells, the cells were treated with 10 mM trypsin for 30 minutes under the same conditions as the cell culture, and then separated from the well. The cells were then added to 100 μL of MTS solution (manufactured by Promega Corp.), and incubated for 30 minutes under the same conditions as the cell culture, and a light absorbance at the wavelength of 490 nm was measured using a plate reader (“iMark Plate Reader” manufactured by Bio-Rad Laboratories, Inc.). The measured light absorbance represents the relative number of the cells.

The average value of the three from the five wells cultured under the same conditions was taken as the measured value. As a control, the wells were used, where the medium of the wells to which DFA III was not added. Because collagen does not dissolve to the amino acid level under these culturing conditions, culturing mixed with collagen was not performed.

The measured values at each point are shown in Table 4 and FIG. 3.

TABLE 4

| | 1 Day after | 4 Day after | 7 Day after | 14 Day after |
|---|---|---|---|---|
| | culture | culture | culture | culture |
| Control | 0.388 | 0.411 | 0.511 | 0.611 |
| 10 mM | 0.309 | 0.323 | 0.388 | 0.736 |
| 50 mM | 0.305 | 0.380 | 0.430 | 0.849 |
| 100 mM | 0.321 | 0.390 | 0.438 | 0.988 |

On day 1, day 4, and day 7 after culturing, the control group exhibited higher values than the experimental group in which DFA III had been added. However, on day 14 after culturing, the group in which DFA III had been added exhibited higher values than the control group, and the values increased as the DFA III concentration increased. These experimental results show that DFA III stimulates cell division of osteoblasts.

### Example 4

### Bone Tissue Analysis by 3D CTR

After preliminary feeding, 12 OVX rats were divided into groups of three rats each. Group A was reared on feed obtained by adding 3% collagen and 5% DFA III to the standard feed; Group B was reared on feed obtained by only adding the same amount of DFA III; Group C was reared on feed obtained by only adding the same amount of collagen; Group D was reared on the standard feed only.

One week, two weeks, three weeks, and one month after the start of feeding, phenobarbital in a dose of 0.5 mg per 10 g of body weight was administered intraperitoneally to anesthetize the rats. Perfusion fixation was performed by injecting 4% paraformaldehyde solution diluted with cacodylate buffer of pH 7.4 and perfusing for 5 minutes from the left ventricle. After fixation, the articular head of the femur and the tibia of the lower limbs were extracted. 3D CTR images of the articular head were taken using a 3D Micro X-ray CTR-mCT2 for experimental animals (manufactured by Rigaku Corp.).

The X-ray images taken after one month are shown in FIG. 4.

In the CTR images, when comparing the groups, Group A has the thickest compact bone and most densely arranged cancellous bone. No marked difference was observed between Group B and Group C. Both compact bone and cancellous bone were thinnest in Group D.

From the 3D CTR images that were taken, five images of cross-sections in the same direction were selected, and the bone area and bone marrow area in each individual was measured and the average taken (Table 5 and FIG. 5).

When comparing each experimental group and the control Group D, the proportion of bone in the articular head was 32.9% increased in Group A, 10.2% increased in Group B, and 8.8% increased in Group C.

TABLE 5

| | Group A | Group B | Group C | Group D |
|---|---|---|---|---|
| Bone area (average) | 420.1 | 320.0 | 290 | 211.4 |
| μm² | | | | |
| Bone marrow | 113.1 | 250.9 | 240.3 | 249.5 |
| area (average) | | | | |
| μm² | | | | |
| Total area | 533.2 | 570.9 | 530.4 | 460.9 |
| μm² | | | | |
| Proportion of bone in | 78.8 | 56.1 | 54.7 | 45.9 |
| articular head (%) | | | | |

From the results above, the group of OVX rats that ingested DFA III or collagen alone had approximately 10% more bone tissue than the group that was fed standard feed alone, and additionally, the group that was fed DFA III and collagen had not less than 30% more bone tissue, demonstrating the synergistic effect of the mixture of DFA III and collagen.

### Example 5

### Confirmation of Recovery Promoting Effect in Case of Osseous Defect

Using 12 OVX rats after preliminary feeding, phenobarbital in a dose of 0.5 mg per 10 g of body weight was administered intraperitoneally to anesthetize the rats. An incision was made in the skin on the upper portion of the knee joint, and then the knee joint was exposed, and a hole approximately 0.5 mm in diameter was made using a dental round bur in the epiphyseal area of the tibia, and then the incision was sutured.

After the surgery, the rats were divided into four groups of three rats each. Group A was reared on feed obtained by adding 3% collagen and 5% DFA III to the standard feed; Group B was reared on feed obtained by only adding the same amount of DFA III; Group C was reared on feed obtained by only adding the same amount of collagen; Group D was reared on the standard feed only.

After one month of feeding, the same anesthesia was performed, and perfusion fixation was performed with 4% paraformaldehyde from the left ventricle in the same manner as the femur experiment. After fixation, the region where the surgery was performed was extracted, and after washing with 0.8 M cacodylate buffer adjusted to pH 7.4, 3D CTR images were taken using the CTR apparatus mentioned above (FIG. 6).

The bone area and the bone marrow area were measured using five images of the osseous defect in the same manner as in Example 4 (Table 6).

TABLE 6

| | Group A | Group B | Group C | Group D |
|---|---|---|---|---|
| Bone area | 238.4 | 90.7 | 90.1 | 80.5 |
| μm² | | | | |
| Bone marrow area | 584.8 | 704.8 | 724.8 | 1099.0 |
| μm² | | | | |
| Total area | 823.2 | 795.5 | 814.8 | 1179.5 |
| μm² | | | | |
| Proportion of bone | 29.0 | 11.4 | 11.1 | 6.8 |
| in all bone (%) | | | | |

The proportion of new bone formation compared to the control Group D was 22.2% greater in Group A, 4.6% greater in Group B, and 4.3% greater in group C.

These results also demonstrate a synergistic effect of the mixture of DFA III and collagen, which is greater than the effect of DFA III or collagen alone.

### INDUSTRIAL APPLICABILITY

The method for promoting osteogenesis of the present invention can be used in the prevention and treatment of osteoporosis.

### BRIEF DESCRIPTION OF DRAWINGS

FIG. 1 shows 2D CTR images of the central portion of the tibia.

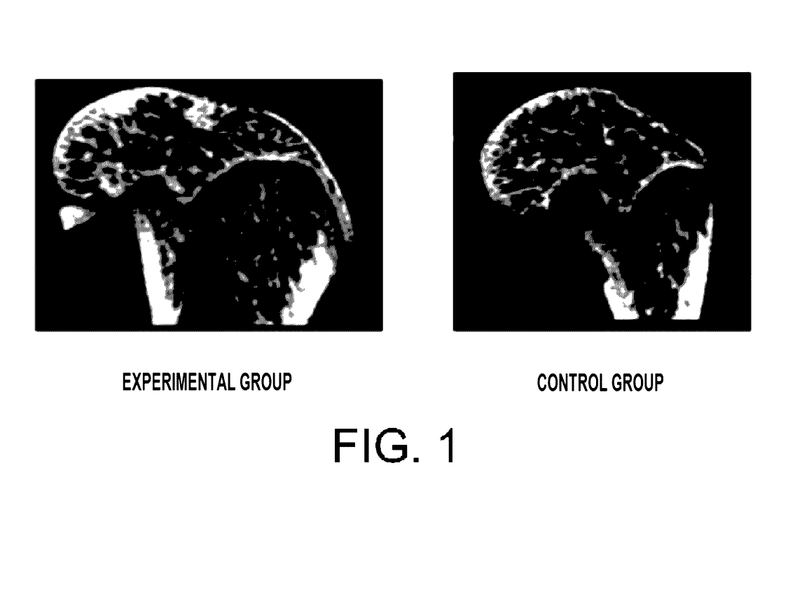

FIG. 2 is a graph of bone mineral density (g/cm²) of the radial bone measured by ultrasound.

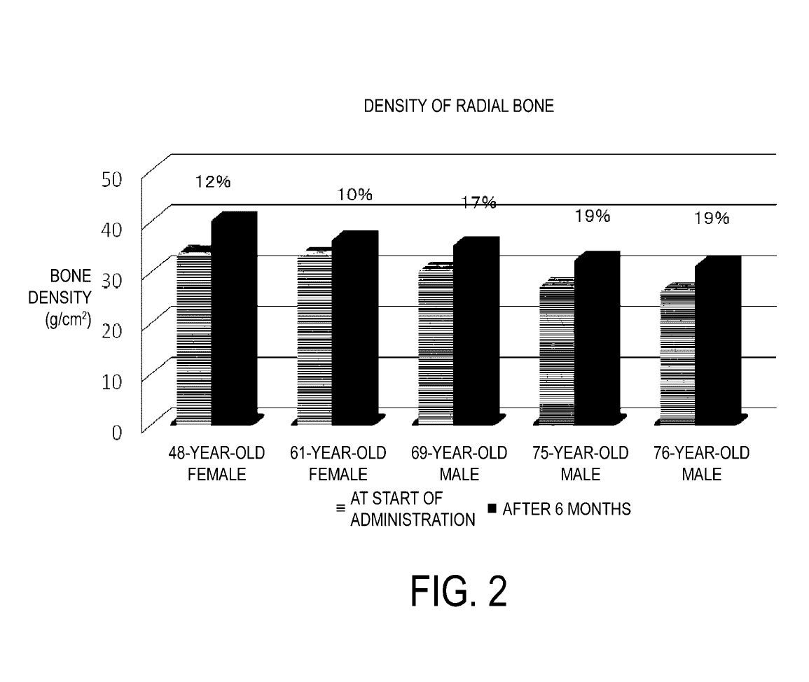

FIG. 3 is a graph of MC3T3-E1 cell culture results (number of cells).

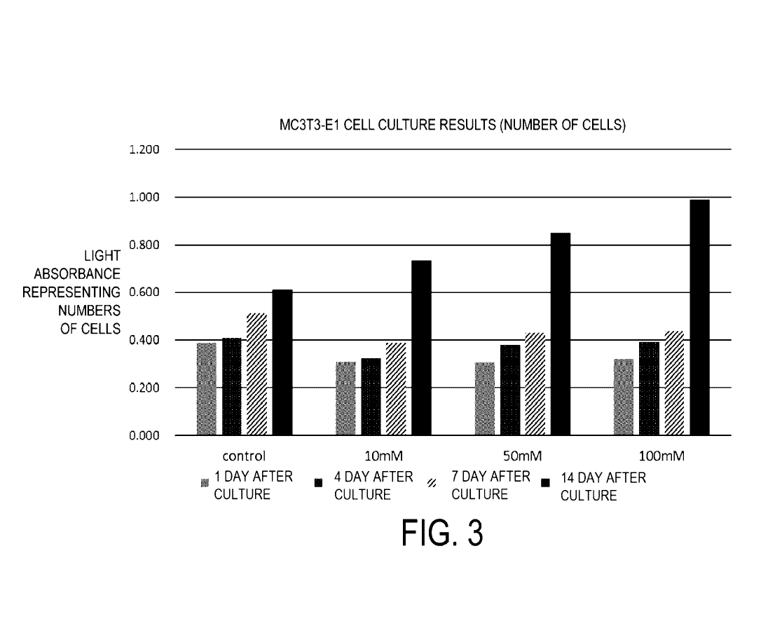

FIG. 4 shows X-ray images of the articular head of the femur one month after the start of feeding.

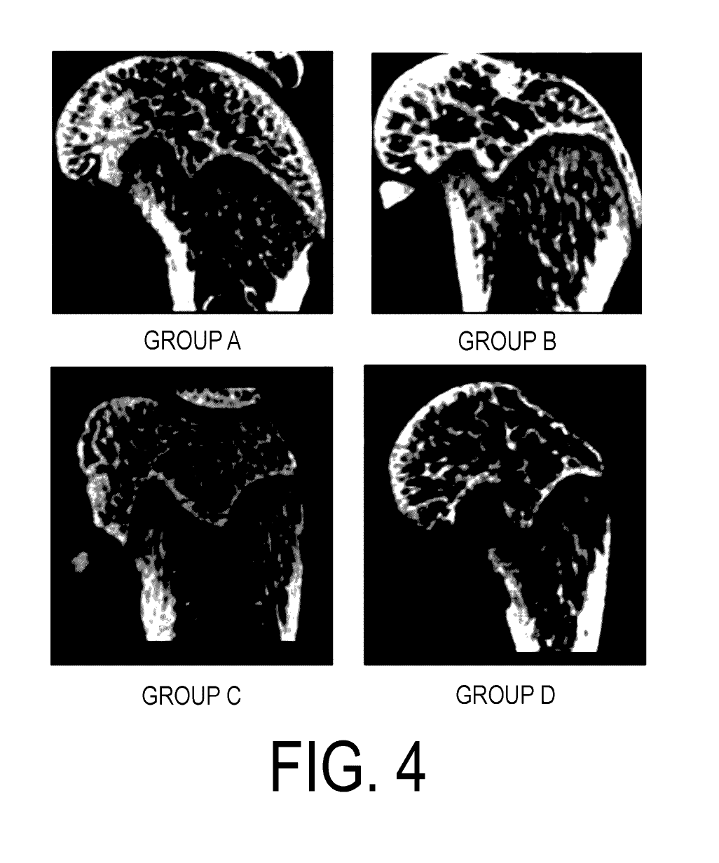

FIG. 5 is a graph of the proportion of bone in articular head one month after the start of administration.

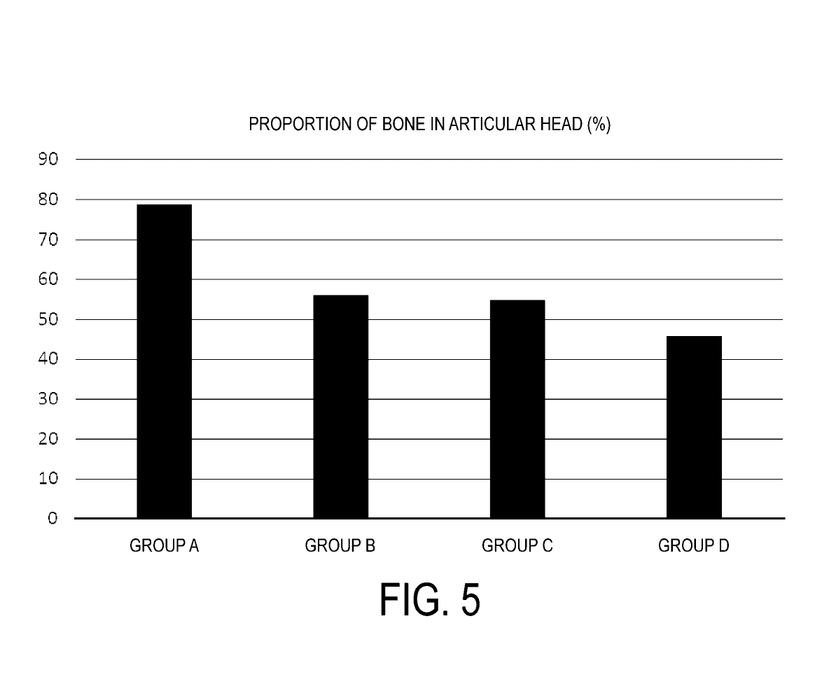

FIG. 6 shows X-ray images after one month of feeding after creating an osseous defect.

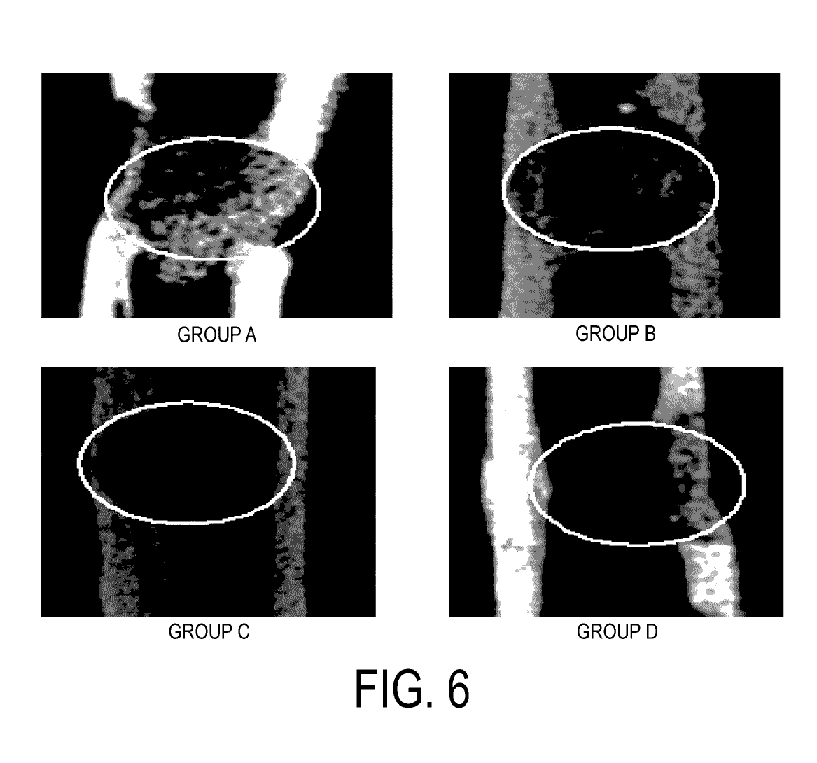

1. A method for promoting osteogenesis, the method comprising:
administering a therapeutically effective amount of collagen and a calcium absorption promoter to a subject.

2. The method according to claim 1, wherein the calcium absorption promoter is difructose anhydride III.

3. The method according to claim 2, wherein the amount of difructose anhydride III is from approximately 1 mg to approximately 50,000 mg.

4. The method according to claim 3, wherein the amount of difructose anhydride III is from approximately 100 mg to approximately 1,000 mg.

5. The method according to claim 1, wherein the amount of collagen is from approximately 100 mg to approximately 80,000 mg.

6. The method according to claim 5, wherein the amount of collagen is from approximately 1,000 mg to approximately 10,000 mg.

7. A method for promoting osteogenesis, comprising:
administering a composition comprising a therapeutically effective amount of collagen and a calcium absorption promoter as active ingredients to a subject.

8. The method according to claim 7, wherein the calcium absorption promoter is difructose anhydride III.

9. The method according to claim 8, wherein a content of difructose anhydride III in the composition is from approximately 0.1 to approximately 30% by weight.

10. The method according to claim 9, wherein the content of difructose anhydride III in the composition is from approximately 0.5 to approximately 20% by weight.

11. The method according to claim 7, wherein a content of collagen in the composition is from approximately 0.1 to approximately 30% by weight.

12. The method according to claim 11, wherein the content of collagen in the composition is from approximately 0.5 to approximately 20% by weight.

13. The method according to claim 7, wherein the administration is oral administration.

14. The method according to claim 7, wherein the composition is a tablet.

15. The method according to claim 7, wherein the composition is a supplement.
